(19) United States
(12) Patent Application Publication
(10) Pub. No.: US 2017/0281193 A1
(43) Pub. Date: Oct. 5, 2017

(54) CLOSURE AND ABLATION OF BODY VISCERA AND CONDUITS

(71) Applicant: Mayo Foundation for Medical Education and Research, Rochester, MN (US)

(72) Inventors: Samuel J. Asirvatham, Rochester, MN (US); David R. Holmes, Jr., Rochester, MN (US)

(73) Assignee: Mayo Foundation for Medical Education and Research, Rochester, MN (US)

(21) Appl. No.: 15/505,982

(22) PCT Filed: Aug. 26, 2015
(86) PCT No.: PCT/US15/46922

§ 371 Date: Feb. 23, 2017

Related U.S. Application Data
(60) Provisional application No. 62/041,790, filed on Aug. 26, 2014.

(51) Int. Cl. A61B 17/12; A61B 18/14

(52) U.S. Cl. CPC A61B 17/12122; A61B 17/12172; A61B 17/12177; A61B 18/1492; A61B 2018/0016

(57) ABSTRACT

Devices and methods can be used to treat heart conditions. Some such devices include endocardial occlusion and ablation devices. For example, such devices can be used for closing and ablating the left atrial appendage to treat atrial fibrillation and to reduce the potential for embolic stroke. In addition, this document provides devices and methods for closing and ablating other body viscera, conduits, valves, and the like.

### CROSS REFERENCE TO RELATED APPLICATIONS

This application claims the benefit of U.S. Provisional Ser. No. 62/041,790 filed Aug. 26, 2014. The disclosure of the prior application is considered part of (and is incorporated by reference in) the disclosure of this application.

### BACKGROUND

### 1. Technical Field

This document relates to devices and methods for the treatment of heart conditions. For example, this document relates to devices and methods for closing and ablating the left atrial appendage to treat atrial fibrillation and to reduce the potential for embolic stroke. In addition, this document relates to devices and methods for closing and ablating other body viscera, conduits, valves, and the like.

### 2. Background Information

Some cardiac structures such as atrial appendages can contribute to blood flow irregularities, which can be associated with various cardiac-related pathologies. For example, complications caused by blood flow irregularities within the left atrial appendage (LAA) and associated with atrial fibrillation can contribute to embolic stroke.

The LAA is a muscular pouch extending from the anterolateral wall of the left atrium of the heart. The LAA serves as a reservoir for the left atrium. During a normal cardiac cycle, the LAA contracts with the left atrium to pump blood from the LAA, which generally prevents blood from stagnating within the LAA. However, during cardiac cycles characterized by arrhythmias (e.g., atrial fibrillation), the LAA may fail to adequately contract. In result, blood may stagnate within the LAA. Stagnant blood within the LAA is susceptible to coagulating and forming a thrombus, which can dislodge from the LAA and ultimately result in an embolic stroke.

Atrial fibrillation is an irregular and often rapid heart rate that commonly causes poor blood flow to the body. During atrial fibrillation, the heart's two upper chambers (the atria) beat chaotically and irregularly—out of coordination with the two lower chambers (the ventricles) of the heart. Atrial fibrillation symptoms include heart palpitations, shortness of breath, and weakness.

Ablation procedures are a treatment for arrhythmia such as atrial tachycardia, atrial flutter, and atrial fibrillation. Energy is delivered from an ablation device to the endocardial and myocardial tissue. The energy delivered causes scarring of the tissue. The scars block impulses firing from within the tissue, thereby electrically “disconnecting” them or “isolating” them from the heart. In some cases, ablation procedures can thereby provide restoration of normal heart rhythms.

### SUMMARY

This document provides devices and methods for the treatment of heart conditions. For example, this document provides devices and methods for occluding and ablating the LAA to treat atrial fibrillation and to reduce the potential for embolic stroke.

In one implementation, an implantable medical device includes a frame comprising multiple elongate elements, a covering attached to the frame, a plurality of electrode pairs fixedly disposed around an outer periphery of the medical device, and at least one pair of distally-located electrodes attached to the frame. The frame is reconfigurable between a low-profile delivery configuration and an expanded configuration. The covering comprises a skirt that restricts blood flow through at least a portion of the frame. The electrode pairs are configured to deliver ablation energy.

Such an implantable medical device may optionally include one or more of the following features. The implantable medical device may further comprise one or more anchor features extending outward from the outer periphery of the medical device. The skirt may be selectively deployable such that at least a portion of the skirt is extendable outward from the outer periphery of the medical device. The covering may be pleated or elastic to facilitate deployment of the selectively deployable skirt. The at least one pair of distally-located electrodes may be configured for pacing or electrographic detection. The plurality of electrode pairs may include two or more different sizes of electrode pairs.

In another implementation, an implantable medical device includes a frame comprising one or more elongate elements, and a covering attached to the frame. The frame is reconfigurable between a low-profile delivery configuration and an expanded configuration. The covering comprises a skirt, and the skirt is selectively deployable such that at least a portion of the skirt is extendable outward from an outer periphery of the medical device.

Such an implantable medical device may optionally include one or more of the following features. The implantable medical device may further comprise one or more anchor features extending outward from the outer periphery of the medical device. The skirt may be selectively deployable such that at least a portion of the skirt is extendable outward from the outer periphery of the medical device. The covering may be pleated or elastic to facilitate deployment of the selectively deployable skirt. The medical device may be selected from a group consisting of: an occluder, a prosthetic valve, a stent, and a filter.

In another implementation, a method for treating a human heart of a patient includes deploying an implantable medical device within a left atrial appendage of the heart, delivering ablation energy from the plurality of electrode pairs to the left atrial appendage, and detecting, using the pair of distally-located electrodes, the presence or absence of an electrogram on the left atrial appendage. The implantable medical device includes a frame comprising multiple elongate elements, a covering attached to the frame, a plurality of electrode pairs fixedly disposed around an outer periphery of the medical device, and at least one pair of distally-located electrodes attached to the frame. The frame is reconfigurable between a low-profile delivery configuration and an expanded configuration. The covering comprises a skirt that restricts blood flow through at least a portion of the frame. The electrode pairs are configured to deliver ablation energy.

Such a method for treating a human heart of a patient may optionally include one or more of the following features. The method may further comprise individually modulating ablation energy delivered to individual pairs of the plurality of electrode pairs. The delivering ablation energy may at least partially continue until one or more gaps between the implantable medical device and the left atrial appendage are determined to be sealed by formation of scar tissue. The skirt may be selectively deployable such that at least a portion of the skirt is extendable outward from the outer periphery of the medical device. The method may further comprise deploying one or more portions of the selectively deployable skirt. The deploying may comprise delivering an energy from outside of the patient that is received by the implantable medical device. The energy may be RF.

Particular embodiments of the subject matter described in this document can be implemented to realize one or more of the following advantages. In some embodiments, the LAA occlusion devices provided herein are configured to deliver ablation energy. As such, a single device can electrically isolate and occlude the LAA to treat atrial fibrillation and to reduce the potential for embolic stroke. In some implementations, the application of ablation energy causes fibrosis of the surrounding tissue leading to an improved seal between the occlusion device and the LAA. In some embodiments, an ablation energy control algorithm can be used to indicate when such leaks between the occlusion device and the tissue are mitigated by the formation of fibrosis from the ablation. Using this method of sealing can provide a good seal without having to substantially stretch tissue, which can lead to compression of coronary arteries. The algorithm can also indicate when an electrode is adjacent to a coronary artery, so that damage to the artery can be avoided. In some embodiments, the location and size of the electrodes on the occlusion devices are selected to prevent aneurysmal dilation at the LAA ostium or loss of integrity of the myocardium that could allow migration of the occlusion devices. In some embodiments, a distal electrode pair is included that can be used for indicating when the LAA is electrically isolated as a result of the ablation. In some embodiments, the heart conditions can be treated in a minimally invasive fashion using the devices and methods provided herein. Such minimally invasive techniques can reduce recovery times, patient discomfort, and treatment costs.

An additional advantage is provided in some embodiments by a selectively deployable skirt that may be included in the occlusion devices provided herein, and in other implantable medical devices. The selectively deployable skirt can be used to achieve a seal between the occlusion devices and the surrounding tissue, including when the topography of the surrounding tissue is irregular such that gaps would otherwise exist between the tissue and the occlusion devices. Such a selectively deployable skirt can also be used as part of various other types of implantable devices such as prosthetic valves, embolic filters, stents, and other devices that benefit from a positive seal between the device and surrounding tissue.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is partial cross-sectional side view showing an occluder device that is deployed in an LAA.

FIG. 2 is a partial cross-sectional side view of the occluder device of FIG. 1 showing a gap between the occluder device and the wall of the LAA.

FIG. 3A is an enlarged view of the area of the gap between the occluder device and the wall of the LAA shown in FIG. 2.

FIG. 3B is the same area shown in FIG. 3A with the addition of scar tissue created by ablation energy delivered from the occlusion device and causing a seal between the occluder device and the wall of the LAA.

Like reference numbers represent corresponding parts throughout.

### DETAILED DESCRIPTION

This document provides devices and methods for the treatment of heart conditions. For example, this document provides devices and methods for occluding and ablating the LAA to treat atrial fibrillation and to reduce the potential for embolic stroke. The devices and methods provided herein may also be used to treat other conditions. Such an approach may be helpful in any tubular structure where there is a gap between a deployed structure (such as a stent) and the wall of the tubular structure. One such example would be the use of a stent to treat esophageal reflux. In this case there may be residual backward flow of gastric fluid which negates some of the advantages of the stent. Deploying a skirt to fill that gap or applying energy to cause the tubular tissue to remodel and constrict around the stent. Such a device could be used in vascular as well as non-vascular structures to treat abnormal flow patterns, such as genito-urinary reflex from the bladder back into the ureters. Such a device may also be useful to prevent cardiac valve regurgitation. Other usages include, but are not limited to, valves including stent valves, pharyngeal devices for sleep apnea, the right atrial appendage, the biliary and pancreatic apparatus, and valved conduits for a percutaneous gastrojejunostomy or gastroduodenostomy for the management of obesity.

The concepts provided herein may also be used in the context of transcatheter valve implants. One such embodiment uses a deflectable catheter for valve delivery that has electrodes protected from the distal circulation (with or without a skirt) with ablation being used to weld and promote fibrosis so as to prevent future paravalvular leaks, without the need for over-distension or tissue rupture and possible distal calcific or other embolization.

Percutaneously deployable combination occlusion and ablation devices are provided herein (also referred to hereinafter as “occluder devices”). In one example implementation, such an occluder device can be used to treat atrial fibrillation and to reduce the potential for embolic stroke. For example, in some implementations the occluder device can be deployed in an LAA. Using a single combination occlusion and ablation device, the LAA can receive ablation energy and the LAA cavity can be sealed off from the rest of the heart.

In some implementations, the ablation energy delivered by the occluder device can electrically isolate the LAA to treat atrial fibrillation. Various types of ablation mechanisms can be used in conjunction with the occluder device. While the examples provided herein may use radiofrequency (RF) energy, it should be understood that the ablation techniques can also incorporate modalities such as, but not limited to, heat, cryogenic substances, high intensity focused ultrasound, lasers, microwaves, and the like, and combinations thereof.

In some embodiments, the occluder device includes electrodes (e.g., distally-located electrodes) that can be used to determine whether the application of ablation energy to the LAA by the occluder device has successfully electrically isolated the LAA to a desired extent. For example, the distally-located electrodes can be used to measure the electrogram on the LAA. A loss of the electrogram on the LAA would indicate electrical isolation of the LAA. In addition, pacing can be provided via the distally-located electrodes to confirm whether conduction from the LAA to the left atrium has been prevented by the ablation. Using those techniques, bi-directional electrical isolation of the LAA can be confirmed using the distally-located electrodes.

In some embodiments, the ablation energy delivered by the occluder device is controlled in a manner that prevents or reduces the risks of creating unintended damage to nearby structures, such as coronary arteries. For example, in some embodiments a single occluder device may include ablation electrodes of two or more different sizes. In some such embodiments, while applying a consistent source of energy to each of the electrodes, more ablation power is naturally delivered by the larger electrodes than by the smaller electrodes. The larger electrodes can be located on the occluder device in locations that make contact with tissue that is capable of receiving more ablation power, whereas the same smaller electrodes can be located on the occluder device in locations that make contact with regions that are more sensitive to unintended damage from ablation.

Some embodiments include additional ways of controlling ablation energy to prevent unintended tissue damage, while providing effective electrical isolation results. For example, in some embodiments each electrode pair can be monitored and controlled separately. That is, ablative power can be modulated differentially to individual electrodes in response to monitored feedback such as, but not limited to, temperature, impedance, and the like. In one example, when the impedance associated with a particular pair of electrodes is low (or falling), while the power being delivered by the pair of electrodes is plateaued at a maximum level, such a combination of factors may indicate that cooling is taking place in the area of the electrodes. That cooling may be the result of a nearby coronary artery in some cases. Therefore, in such a case the ablation power delivered to the particular pair of electrodes can be modulated to a lower level (or turned off) so as to prevent damage to the nearby coronary artery.

In some implementations, additional or alternative measures can be taken to protect coronary arteries from damage due to ablation of the LAA by the occluder devices provided herein. For example, in some implementations angiography can alternatively or additionally be used to identify locations of coronary arteries. Also alternatively or additionally, in some implementations a catheter can be placed in the coronary sinus and cooling media (e.g., cooled blood) can be introduced therein to protect coronary arteries during ablation of the LAA. Also alternatively or additionally, in some implementations a Doppler element can be included to detect blood flow to identify locations of coronary arteries. When the locations of coronary arteries are so identified, the ablation energy delivered by the electrodes in the vicinity of the coronary arteries can be modulated to prevent damage to the coronary arteries. Also, when arteries are nearby, increased power delivery can be made in some cases because the cooling from the artery is self-protective, and this would allow sealing from impedance change and fibrosis exactly where pressure should not be applied.

During ablation, if the temperature does not increase but impedance drops or is phasic, consistent with nearby blood flow, then power delivery can be titrated, given the proximity of a coronary vessel. In addition, the pressure of expansion can also be titrated to avoid compression of the artery. Similarly, when there is no concern of a nearby vessel near the electrodes which are insulated and covered in the direction toward the left atrium, high energy delivery to purposely produce an impedance increase and coagulum to promote fibrosis and closure can be done without fear of particulate embolization.

In addition to delivering ablation energy to electrically isolate the LAA, in some cases ablation energy may also be delivered to enhance the seal between the occluder device and the surrounding tissue, and/or to enhance the anchoring (migration resistance) between the occluder device and the surrounding tissue. In some implementations, gaps between the occluder device and the surrounding tissue may exist when the occluder device is deployed in an LAA. That may be the result when, for example, the shape of the LAA is irregular to the extent that the occluder device is unable to fully conform to the irregular tissue topography around the occluder device. When ablation energy is applied from the occluder device in the area of such gaps, scar tissue may form so as to occlude the gaps. In effect, a positive remodeling of the LAA tissue may occur as a result of the ablation energy from the occluder device. In that manner, the seal between the occluder device and the surrounding tissue can be enhanced by the delivery of ablation energy from the occluder device.

When delivering ablation energy to seal gaps between the occluder device and the surrounding tissue, the ablation energy delivered can be monitored and controlled in a manner that prevents or reduces the risks of creating unintended damage to the tissue and sensitive nearby structures. For example, by monitoring the impedance associated with individual electrode pairs during the delivery of ablation energy to seal gaps, the development of scar tissue in the area of such gaps can be detected. More particularly, when a gap exists in the area of an electrode pair between the occluder device and the surrounding tissue the initial impedance associated with the electrode pair will be low. As the scar tissue forms as a result of the ablation energy, the impedance associated with the electrode pair will rise. The rise in impedance can be detected, and the ablation energy delivered by the particular electrode pair (or all electrode pairs) can be reduced or discontinued in response.

In some embodiments of the implantable devices described herein, the devices and the electrodes used for ablation can be additionally or alternatively be used for permanent pacing or defibrillation. In one such example, the electrodes used for ablation can be used for defibrillation around the LAA.

It should be understood that in some cases the devices described herein need not be permanently implanted. Rather, in some implementations the devices are temporarily used to modify tissue such as to treat damaged, prolapsing, regurgitant, or otherwise dysfunctional elements or structures in a viscus or conduit. For example, in some embodiments a partial circumferential (e.g., an ovular segment) embodiment of the devices described herein can be used along the mitral valve annulus. Such a device can include components to have electrodes approximated with prolapsing or redundant leaflets to shrink the leaflets, and/or to cause temporary adhesion of the leaflet portions to each other facilitated by the application of RF energy or other electrical energy sources.

In some embodiments, a selectively deployable skirt (or one or more portions of a skirt) may be included in the occluder devices provided herein. When the occluder devices are deployed from a delivery sheath, the occluder devices expand to a nominal diameter (which is, in general, a diameter that is determined by the size of the LAA). However, in some circumstances one or more gaps may exist between the nominal diameter of the occluder device and the tissue surfaces surrounding the occluder device. The selectively deployable skirt can be used to achieve a seal between the occlusion device and the surrounding tissue, including when the topography of the surrounding tissue is irregular such that gaps exist in particular places between the tissue and the occlusion devices.

In some embodiments, some portions of the selectively deployable skirt can be selectively actuated to radially expand the skirt while other portions of the selectively deployable skirt that are not so actuated remain at the nominal diameter. In some embodiments, all portions of the selectively deployable skirt are actuated as a unit. Skirt deployment may be affected using mechanisms such as, but not limited to, mechanical, energy (e.g., RF, heat, etc.), shape-memory materials, and the like. For example, in some embodiments an adhesive is used to restrain a portion of the selectively deployable skirt, and the adhesive can be deactivated to release the portion of the selectively deployable skirt to expand. In some embodiments, the selectively deployable skirt can be remotely activated. That is, activation can be performed external to the patient's body.

In some embodiments, deployment of the skirt is passive such that the skirt can billow outward after deployment. In some embodiments, the skirt includes pleats or creases that can allow the skirt to expand. In some embodiments, the skirt is elastic to allow the skirt to expand. Such a selectively deployable skirt can also be used as part of various other types of implantable devices such as prosthetic valves, embolic filters, stents, and other devices that benefit from a positive seal between the device and surrounding tissue.

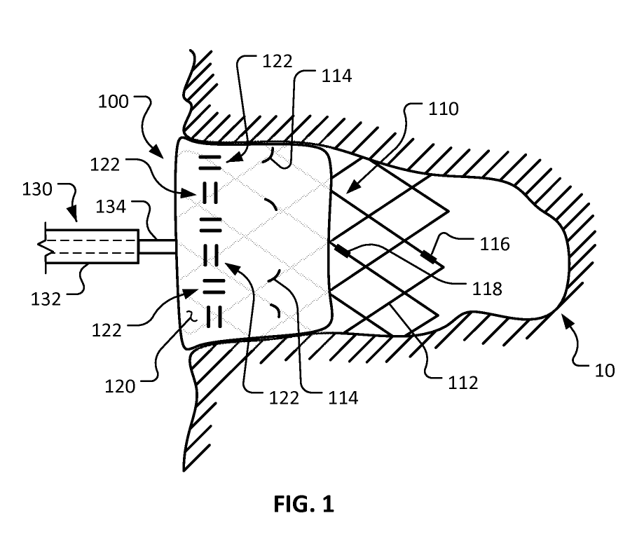

Referring to FIG. 1, an example occluder device 100 can be implanted to treat an LAA 10. While the implant orientation as depicted has the proximal end of occluder device 100 generally flush with the ostia of LAA 10, in some implementations occluder device 100 is implanted further within LAA 10 or further outside of LAA 10.

In the depicted example, occluder device 100 is delivered to the site of LAA 10 using a delivery system 130. Delivery system 130 includes a sheath 132 and a catheter 134. Catheter 134 is slidably disposed within sheath 132, and is releasably coupled to occluder device 100. In the depicted orientation, occluder device 100 is in an expanded or partially expanded configuration. Prior to being expanded at the site of LAA 10, occluder device 100 is in a low-profile configuration and contained within the lumen of sheath 132. In some embodiments, the fully expanded size of occluder device 100 is larger than LAA 10 such that occluder device 100 substantially fills (and may slightly stretch) LAA 100.

In some implementations, a transesophageal echocardiogram (TOE) is performed to measure LAA 10 to determine which size occluder device 100 to be implanted. In some implementations, a guidewire (not shown) is first percutaneously inserted into the patient's vasculature (e.g., via a femoral artery, radial artery, etc.). X-ray fluoroscopy can be used to visualize the navigation of the guidewire and successive devices within the patient's body. Various radiopaque (RO) markers can be included on deliver system 130 and occluder device 100 to enhance their radiographic visibility. The inter-atrial septum can be crossed using a trans-septal access system, and delivery system 130 containing occluder device 100 can be advanced over the guidewire into the left atrium toward LAA 10. Occluder device 100 is then deployed into LAA 10 by distally translating catheter 134 in relation to sheath 132. Occluder device 100 is thereby made to emerge from sheath 132, and occluder device 100 expands within LAA 10 to the configuration shown. In some implementations, as explained further below, ablation energy can then be delivered from occluder device 100 to LAA 10. Prior to releasing occluder device 100 from catheter 132, release criteria can be confirmed via instruments and modalities such as fluoroscopy, the ablation control system, electrogram, and the like.

Occluder device 100 includes a frame 110 and a skirt 120. In some embodiments, skirt 120 is disposed on the outside of at least a portion of frame 110. In some embodiments, skirt 120 is disposed on the inside of at least a portion of frame 110. In some embodiments, skirt 120 is disposed on the inside or the outside of substantially the entire frame 110.

In the depicted embodiment, frame 110 is constructed of multiple elongate elements 112. In some embodiments, the diameter or thickness of multiple elongate elements 112 may be within a range of about 0.008″ to about 0.015″ (about 0.2 mm to about 0.4 mm), or about 0.009″ to about 0.030″ (about 0.23 mm to about 0.8 mm), but in other embodiments elongate elements 112 having smaller or larger diameters or thicknesses may be used. Elongate elements 112 can be made of metallic or polymeric materials. For example, in some embodiments, elongate elements 112 are made of metallic materials such as, but not limited to, nitinol, stainless steels (e.g., 316L, etc.), alloy L-605, titanium, and the like. The super-elasticity of nitinol can make nitinol an effective choice for the elongate elements 112 to construct frame 110. In some embodiments, elongate elements 112 are multiple wires that are wound together to make frame 110. In some embodiments, frame 110 is constructed by cutting a tube or sheet of material and expanding the material to create the cellular structure of frame 110. For example, in some embodiments a tube of nitinol material is laser cut and then the tube is expanded and heat-set in the expanded configuration. However, other ways of constructing frame 110 are also within the scope of this disclosure.

Skirt 120 can be comprised of a fabric, a membranous material, a film material, and the like. In some embodiments, skirt 120 is made of materials such as, but not limited to, Dacron®, Nylon, TFE, PTFE, ePTFE, and the like. The material of skirt 120 may be engineered to contain cells and cellular products. Some portions or all of skirt 120 can be treated in some embodiments. Such treatments can include, but are not limited to, perforations to modulate fluid flow through skirt 120, and treatments to affect the propensity for tissue ingrowth to skirt 120. In some embodiments, skirt 120 is treated to make skirt 120 stiffer or to add surface texture. For example, in some embodiments skirt 120 is treated with FEP powder to provide a stiffened skirt 120 or roughened surface on skirt 120. Other skirt 120 material treatment techniques can also be employed to provide beneficial mechanical properties and tissue response interactions. In some embodiments, skirt 120 may be chemically modified to promote one or more of endothelial cell attachment, endothelial cell migration, endothelial cell proliferation, or resistance to thrombosis. In some embodiments, skirt 120 is attached to frame 110 by methods such as, but not limited to, stitching, adhering (e.g., FEP), using clips, interweaving, and the like.

Occluder device 100 also includes anchor features 114. Anchor features 114 extend outward from the periphery of skirt 120 and/or frame 110 so as to engage with the wall of LAA 10. Anchor features 114 help to prevent migration of occluder device 100 in relation to LAA 10. In some embodiments, anchor features 114 can include, but are not limited to, barbs, hooks, piercing members, coils, clips, sutures, atraumatic members, and the like. In some embodiments, anchor features 114 are integral with elongate elements 112. In some embodiments, anchor features 114 are attached to elongate elements 112. In some embodiments, anchor features 114 are attached to skirt 120.

Occluder device 100 also includes multiple electrode pairs 122 that can deliver ablation energy. Electrode pairs 122 are disposed around the circumference of occluder device 100. Electrode pairs 122 are separated from anchor features 114. The separation can help to ensure that the tissue with which anchor features 114 engage is not directly affected by ablation (which could cause loss of tissue integrity and a weakened anchorage). In the depicted embodiment, the orientations of adjacent electrode pairs 122 are alternated (radial versus longitudinal) around the circumference of occluder device 100. However, in some embodiments other patterns of orientating electrode pairs 122 are used.

In some embodiments, each electrode pair 122 is individually monitored and controlled. That is, ablative power can be modulated differentially to individual electrode pairs 122 in response to monitored feedback such as, but not limited to, temperature, impedance, and the like. In some embodiments, the ablative power is transmitted to electrode pairs 122 via wires and detachable connections within catheter 134.

In some embodiments, some electrode pairs 122 are larger than other electrode pairs 122. For example, in some such embodiments larger electrode pairs 122 are positioned to make contact with the posterior wall of LAA 10, and smaller electrode pairs 122 are positioned to make contact with the anterior wall of LAA 10. At least one benefit for such an arrangement is that the posterior wall has the left atrial ridge and ablation of that whole ridge can advantageously take care of multiple arrhythmogenic substrates, such as LAA 10 itself, the vein of Marshall, and the anterior wall of the pulmonary vein. Using larger electrode pairs 122 in those locations can provide deeper lesions in the posterior wall location using the same ablation energy source that is used at other sites having smaller electrode pairs 122. Occluder device 100 can include RO indicators that identify the size of electrodes pairs 122 under fluoroscopy, so that the clinician can orientate occluder device 100 in relation to LAA 10 as desired.

In some embodiments, ablation power is delivered initially to all the electrode pairs 122, and such power can thereafter be differentially modulated to individual electrode pairs 122 in accordance with a control algorithm. In some embodiments, the modulation can be based at least in part on how the impedance associated with the individual electrode pairs 122 is dropping. For example, in one control algorithm if the impedance drops the power is automatically increase to a maximum plateau level. Such algorithms can also include, for example, temperature measurements, as well as power and impedance determinations. If the power plateaus at maximum, in some cases it may indicate that the tissue is being cooled externally. For example, when such cooling is occurring, the power may plateau at maximum while the impedance is low or falling. In such a case, it may be advantageous to reduce the power to prevent unintended damage to tissues such as coronary arteries.

In another example, algorithms can be used to help identify and seal leaks (also referred to herein as a “gap”) between occluder device 100 and LAA 10 that exist initially after deployment of occluder device 100 in LAA 10. In such a scenario, the power delivered to a particular electrode pair 122 near a gap may plateau at maximum, the temperature may be low, and the impedance may remain low (because the particular electrode pair 122 is not in contact with the wall tissue of LAA 10). In that case, the algorithm can keep delivering power or may even increase the power. Eventually, scar tissue to seal the leaks may form as a result of the ablation energy delivered to the wall tissue of LAA 10.

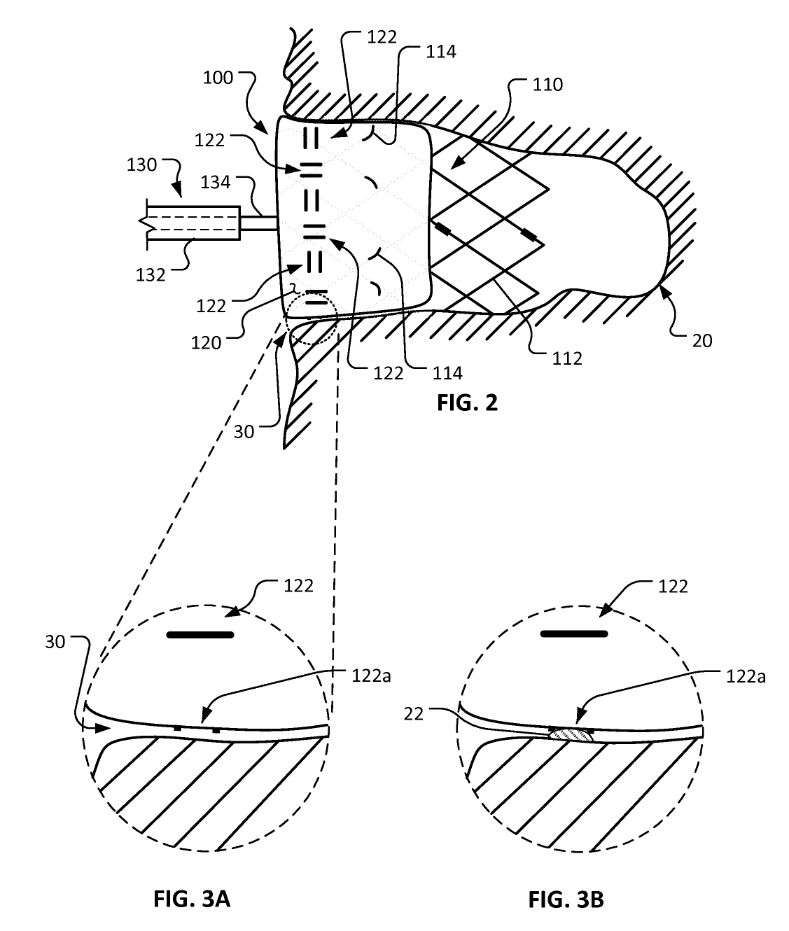

Referring to FIGS. 2, 3A and 3B, occluder device 100 is shown implanted in an LAA 20. In this example, a gap 30 exists between occluder device 100 and LAA 20. In some cases, two or more such gaps 30 may exist initially after deployment of occluder device 100 in LAA 20. Such gaps 30 may be undesirable because, for example, gaps 30 may be a source of thrombus emboli that can cause stroke.

An electrode pair 122a is located on skirt 120 near gap 30. As ablation energy is delivered from electrode pair 122a, fibrosis will occur resulting in scar tissue 22 formed on the wall of LAA 20. Scar tissue 22 may develop in size to seal gap 30, and may enhance the anchoring of occluder device 100 to LAA 20. In some embodiments, the control algorithm for the ablation process can detect when scar tissue 22 has developed to the extent that it seals gap 30 because the impedance associated with electrode pair 122a will increase when scar tissue 22 is in contact with electrode pair 122a.

It should be understood that the occluder device 100, with its capability to seal leaks as described herein, can allow for a looser initial fit in LAA 10 as compared to some other occluder devices that do not include the capability to deliver ablation energy to seal leaks. This feature of occluder device 100 can therefore be advantageous because tighter fits between occluder devices and LAA 10 may lead to undesirable effects including, but not limited to, compression of the circumflex artery.

Referring again to FIG. 1, in some embodiments, occluder device 100 also includes distally-located electrodes 116 and 118. Distally-located electrodes 116 and 118 can be used to determine whether the application of ablation energy to LAA 10 by occluder device 100 has successfully electrically isolated LAA 10. For example, in some embodiments distally-located electrodes 116 and 118 can be used to measure the electrogram on LAA 10. A loss of the electrogram on LAA 10 is an indicator of the electrical isolation of LAA 10. In addition or as an alternative, pacing can be provided via distally-located electrodes 116 and 118 to confirm whether conduction from LAA 10 to the left atrium has been prevented by the ablation. Using those techniques, bi-directional electrical isolation of LAA 10 can be confirmed using distally-located electrodes 116 and 118.

In some embodiments, at least portions (or all) of skirt 120 are selectively deployable to an increased outer periphery. That is, when occluder device 100 is deployed from delivery sheath 130, occluder device 100 expands to a nominal peripheral size (which is, in general, a size that is determined by the size of LAA 10). However, in some circumstances one or more gaps may exist between the nominal peripheral size of occluder device 100 and the tissue surfaces surrounding the occluder device. This may be the case, for example, when the topography of the surrounding tissue is irregular such that gaps exist in particular places between the tissue and occlusion device 100.

In some embodiments, the selectively deployable skirt 120 can be used to seal gaps between occlusion device 100 and the surrounding tissue. For example, in some embodiments some portions of selectively deployable skirt 120 (but not other portions of selectively deployable skirt 120) can be actuated to radially expand portions of skirt 120, while other portions of selectively deployable skirt 120 that are not actuated remain at the nominal size and configuration. In some embodiments, the entirety of selectively deployable skirt 120 is actuated as a unit. That is, the deployable portions of skirt 120 all deploy in unison. In some embodiments, one or more elongate elements 112 of frame 110 are configured to be actuatable to deploy selectively deployable skirt 120.

The deployment of selectively deployable skirt 120 may be actuated using mechanisms such as, but not limited to, mechanical features (e.g., springs, threaded devices, etc.), energy (e.g., RF, heat, microwaves, etc.), shape-memory materials, and the like. For example, in some embodiments an adhesive is used to restrain a portion (or all) of selectively deployable skirt 120, and the adhesive can be deactivated to release selectively deployable skirt 120 to expand.

In some embodiments, deployment of selectively deployable skirt 120 is passive such that skirt 120 can billow outward after deployment.

In some embodiments, selectively deployable skirt 120 includes pleats or creases in the material that can allow skirt 120 to radially expand. In some embodiments, at least portions of selectively deployable skirt 120 are elastic to allow skirt 120 to expand.

Such a selectively deployable skirt 120 can also be used as part of various other types of implantable devices such as prosthetic valves, embolic filters, and other devices that benefit from a positive seal between the device and surrounding tissue. Such devices with selectively deployable skirt 120 are within the scope of this disclosure.

In some cases, a complete seal around an implantable device may not be desirable, such as if a vessel such as a pulmonary vein or mitral valve flow would be blocked. For valves, the selectively deployable skirt may not be deployed unless a paravalvular leak occurs to avoid potential adverse complications such as ventricular arrhythmia.

Differential deployment of a selectively deployable skirt could be achieved by at least the following mechanisms. During a device placement procedure, RF activation could be applied to deploy a selectively deployable skirt. The skirt could be differentially deployed by ablating certain retaining structures of the skirt that are susceptible to ablative energy. In other embodiments, remote activation via energy sources such as, but not limited to, magnetic resonance (MR), focused MR, or ultrasound could be delivered to deploy a selectively deployable skirt at some time (e.g., hours, days, weeks, months, years) after deployment of the implantable device. This concept can be advantageous in some circumstances because, e.g., in the case of an LAA leak, a leak can develop and/or get bigger over the course of time. Rather than go back to do a catheter procedure, the problem could be fixed remotely. Similarly, this could prevent a procedure (catheter or surgery) to repair paravalvular leaks. For remote activation, in some embodiments the skirt would have an element (like a strut) with a known resonant frequency element. Each of these elements or struts would have a different resonant frequency. Then ultrasound can be delivered from an external source at one of the integral harmonics of that resonant frequency for that strut for differential deployment. This configuration would provide a remotely-deployable, portion-specific selectively deployable skirt.

These selectively deployable skirt concepts can also be used in combination with stents for making sphincters for GI, urological, and gynecological work, to provide some further example implementations.

While the devices and the electrodes described herein are mainly described in the context of ablation, it should be understood that the devices and electrodes can be additionally or alternatively be used for permanent pacing or defibrillation. In one non-limiting example, the electrodes used for ablation of the LAA can be used for defibrillation around the LAA.

It should be understood that the devices described herein need not be permanently implanted. Rather, in some embodiments the devices are temporarily used to modify tissue to treat damaged, prolapsing, regurgitant, or otherwise dysfunctional elements or structures in a viscus or conduit. For example, in some embodiments a partial circumferential embodiment of the devices described herein can be used along the mitral valve annulus. Such a device can include components to have electrodes approximated with prolapsing or redundant leaflets to shrink the leaflets, and/or to cause temporary adhesion of the leaflet portions to each other facilitated by the application of RF energy or other electrical energy sources.

In some implementations of the devices described above, the device does not completely occlude the conduit or viscus in which it is deployed. In some embodiments, the devices only partially occlude the conduit or viscus—to, for example, either increase flow velocity or facilitate normal, annular, and valve function. For example, the devices described above for LAA occlusion can be placed across the mitral valve and apposed and secured to the annulus either on the atrial, true annular or ventricular portion, but without occluding the remaining donut-ring shaped structure would facilitate valve approximation and treat patients who have primarily annular dilation. The selectively deployable skirt actuation technique in this implementation would be used to size the gap between the inner and outer diameters of the donut-shaped device that would be kept in place. Similar implementations for other cardiac valves can also be envisioned within the scope of this disclosure.

In some implementations of the devices described above, the device is similar to the LAA closure device with simultaneous ablation, but the skirt material itself (or a portion thereof) is porous and filter-like (not configured for complete occlusion). This device can be placed, for example, in the interior vena cava or other parts of the venous system to prevent pulmonary embolization in patients who are at risk for conditions such as, but not limited to, pulmonary embolization or deep vein thrombosis.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An implantable medical device comprising:
a frame comprising multiple elongate elements, the frame being reconfigurable between a low-profile delivery configuration and an expanded configuration;
a covering, attached to the frame, the covering comprising a skirt that restricts blood flow through at least a portion of the frame;
a plurality of electrode pairs fixedly disposed around an outer periphery of the medical device, the electrode pairs configured to deliver ablation energy; and
at least one pair of distally-located electrodes attached to the frame.

2. The implantable medical device of claim 1, further comprising one or more anchor features extending outward from the outer periphery of the medical device.

3. The implantable medical device of claim 1, wherein the skirt is selectively deployable such that at least a portion of the skirt is extendable outward from the outer periphery of the medical device.

4. The implantable medical device of claim 3, wherein the covering is pleated or elastic to facilitate deployment of the selectively deployable skirt.

5. The implantable medical device of claim 1, wherein the at least one pair of distally-located electrodes are configured for pacing or electrographic detection.

6. The implantable medical device of claim 1, wherein the plurality of electrode pairs include two or more different sizes of electrode pairs.

7. An implantable medical device comprising:
a frame comprising one or more elongate elements, the frame being reconfigurable between a low-profile delivery configuration and an expanded configuration; and
a covering, attached to the frame, the covering comprising a skirt, wherein the skirt is selectively deployable such that at least a portion of the skirt is extendable outward from an outer periphery of the medical device.

8. The implantable medical device of claim 7, wherein the medical device is selected from a group consisting of: an occluder, a prosthetic valve, a stent, and a filter.

9. An implantable medical device system comprising:
a deployment system including a catheter and a sheath defining a lumen, wherein the catheter is slidably disposed within the lumen;
a frame comprising multiple elongate elements, the frame being reconfigurable between a low-profile delivery configuration and an expanded configuration, wherein the frame is in the low-profile deliver configuration when the frame is confined within the lumen and wherein the frame is in the expanded configuration when the frame is not confined in the lumen;
a covering, attached to the frame, the covering comprising a skirt that restricts blood flow through at least a portion of the frame;
a plurality of electrode pairs fixedly disposed around an outer periphery of the medical device, the electrode pairs configured to deliver ablation energy; and
at least one pair of distally-located electrodes attached to the frame.

10. The implantable medical device system of claim 9, further comprising an ablation energy source and controller that runs an algorithm for controlling ablation energy delivered to the plurality of electrode pairs.

11. A method for treating a human heart of a patient, the method comprising:
deploying an implantable medical device within a left atrial appendage of the heart, wherein the implantable medical device comprises:
a frame comprising multiple elongate elements, the frame being reconfigurable between a low-profile delivery configuration and an expanded configuration;
a covering, attached to the frame, the covering comprising a skirt that restricts blood flow through at least a portion of the frame;
a plurality of electrode pairs fixedly disposed around an outer periphery of the medical device, the electrode pairs configured to deliver ablation energy; and
at least one pair of distally-located electrodes attached to the frame;
delivering ablation energy from the plurality of electrode pairs to the left atrial appendage; and
detecting, using the pair of distally-located electrodes, the presence or absence of an electrogram on the left atrial appendage.

12. The method of claim 11, further comprising individually modulating ablation energy delivered to individual pairs of the plurality of electrode pairs.

13. The method of claim 11, wherein the delivering ablation energy at least partially continues until one or more gaps between the implantable medical device and the left atrial appendage are determined to be sealed by formation of scar tissue.

14. The method of claim 11, wherein the skirt is selectively deployable such that at least a portion of the skirt is extendable outward from the outer periphery of the medical device.

15. The method of claim 14, further comprising deploying one or more portions of the selectively deployable skirt.

16. The method of claim 15, wherein the deploying comprises delivering an energy from outside of the patient that is received by the implantable medical device.

17. The method of claim 16, wherein the energy is RF.
